(19) United States
(12) Patent Application Publication
(10) Pub. No.: US 2009/0281548 A1
(43) Pub. Date: Nov. 12, 2009

(54) DEVICE FOR FILLING BONE CAVITIES WITH FLUID CEMENT, ACETABULAR CAVITIES IN PARTICULAR

(76) Inventor: Giovanni Faccioli, Monzambano (MN) (IT)

Correspondence Address: KEUSEY, TUTUNJIAN & BITETTO, P.C., 20 CROSSWAYS PARK NORTH, SUITE 210, WOODBURY, NY, 11797, US

(21) Appl. No.: 12/440,610

(22) PCT Filed: Sep. 17, 2007
(86) PCT No.: PCT/IB07/02676

§ 371 Date: Mar. 10, 2009

(30) Foreign Application Priority Data
Sep. 18, 2006 (EP) 06425641.5

(51) Int. Cl. A61B 17/58

(52) U.S. Cl. 606/93

(57) ABSTRACT

A device for filling bone cavities with fluid cement, in particular, acetabular cavities, includes a transfer channel for transferring a fluid cement with a first opening which is associated to a dispenser instrument for dispensing the fluid cement and a second opening through which the fluid cement exits, and that is associated with an applicator element of the fluid to a bone cavity. The applicator element has a useful dispensing surface that is larger than the transfer channel and includes a substantially plate-like body whose main opposite surfaces define a first side for connecting to the transfer channel and a second side for the application of the fluid cement. The second side has a containing border of the fluid cement which is positionable in contact against the edge of the bone cavity and which has a perimeter turned-up edge of the plate-like body which extends crosswise from the body.

The present invention refers to a device for filling bone cavities with fluid cement, acetabular cavities in particular.

As is common knowledge, arthroplasty requiring the implant of a prosthetic device to a bone or joint of human body, normally needs a certain quantity of cement (acrylic resins or the like) to keep the prosthesis permanently in place. In many different types of implant, bone cement is injected using a syringe which can be mounted on a dispensing gun containing the fluid cement and has a needle, a rigid cannula or the like, for its application.

After having inserted the free end of the cannula inside the cavity that needs filling, the fluid cement is injected directly in situ with the syringe.

While this is a particularly practical and functional way to fill-in femoral, humeral and vertebral cavities or the like, this equipment is found to be inefficient if bone cement needs to be injected into acetabular cavities for the subsequent application of hip prosthesis.

For this type of operation, the spherical head of the hip prosthesis has to be fitted inside the acetabular cavity of the patient, interposing a layer of fluid cement which, once hardened, becomes the bed for the prosthesis joint.

As a rule this type of implant requires the work of a medical operator who manually shapes a mass of cement at the doughy state and introduces it inside the acetabular cavity.

The mass of cement is then compacted inside the cavity with special hammering instruments, with a hammering side shaped like a dome.

Thanks to the particular shape of the hammering instruments the mass of cement is modelled to form a substantially spherically shaped niche on it. This known type of implant does, however, have a few drawbacks.

The fact that the mass of cement is handled manually, for instance, exposes the medical operator to direct contact with potentially toxic and reactive substances. Moreover, exposure of the cement to the external environment can easily compromise its sterility, rendering it a dangerous vehicle for transmitting infections to the patient undergoing the operation.

It is also necessary to stress the fact that this operation is rather awkward and tiring both for the medical operator and the patient.

To prevent such drawbacks, the use of particular devices is known that consist of a syringe the dispensing end of which is widened and can be placed in contact with the edge of the acetabular cavity to prevent the cement from leaking out of the cavity itself.

Such devices are known in GB 2 104 390, WO 01/10356, EP 0 650 707, WO 02/30339 and U.S. Pat. No. 5,741,265.

However, even these devices have several drawbacks one of which being that they do not allow an easy and practical use of the known type of fluid cement. In this regard the fact is underlined that, in order for the fluid cement to come out of the syringe and, at the same time, set firmly inside the acetabular cavity, it must be of a pre-established viscosity.

Taken into account the fact that the viscosity of the traditional fluid cement increases rapidly over time, it is necessary that the medical operator using the syringe is able to assess the viscosity state of the fluid cement practically, easily and with sufficient accuracy, which is not possible to get with the known type of devices.

The main aim of this invention is to provide a device for filling bone cavities with fluid cement, acetabular cavities in particular, allowing to introduce bone cement under optimally sterile and safe conditions both for the medical operator who is doing the operation and for the patient who is undergoing it.

A further object of this invention is to allow to fill bone cavities with fluid cement in a quick, practical, easy and functional way, without any serious difficulties for the medical operator and great discomfort for the patient.

Yet another object of this invention is to provide a device that will overcome the above mentioned drawbacks of the well known technique, with a simple, rational and cost-effective solution.

The objects mentioned above are all achieved by this device for filling bone cavities with fluid cement, acetabular cavities in particular, comprising at least one transfer channel for transferring a fluid cement with at least one first opening which can be associated to a dispenser instrument for dispensing said fluid cement and a second opening through which said fluid cement exits, and at least one applicator element of said fluid cement to a bone cavity which can be associated to said transfer channel by said second opening and has a useful dispensing surface that is larger than said transfer channel, characterised in that said applicator element comprises a substantially plate-like body whose main opposite surfaces define a first connecting side to said transfer channel and a second application side of said fluid cement, said second side comprising a containing border of said fluid cement which can be positioned in contact against the edge of said bone cavity and which comprises a perimeter turned-up edge of said plate-like body which extends substantially crosswise from the body itself.

Further characteristics and advantages of this invention will appear even more evident from the detailed description of a preferred, but not exclusive, embodiment of a device for filling bone cavities with fluid cement, acetabular cavities in particular, illustrated by way of non limiting example in the accompanying drawings, wherein:

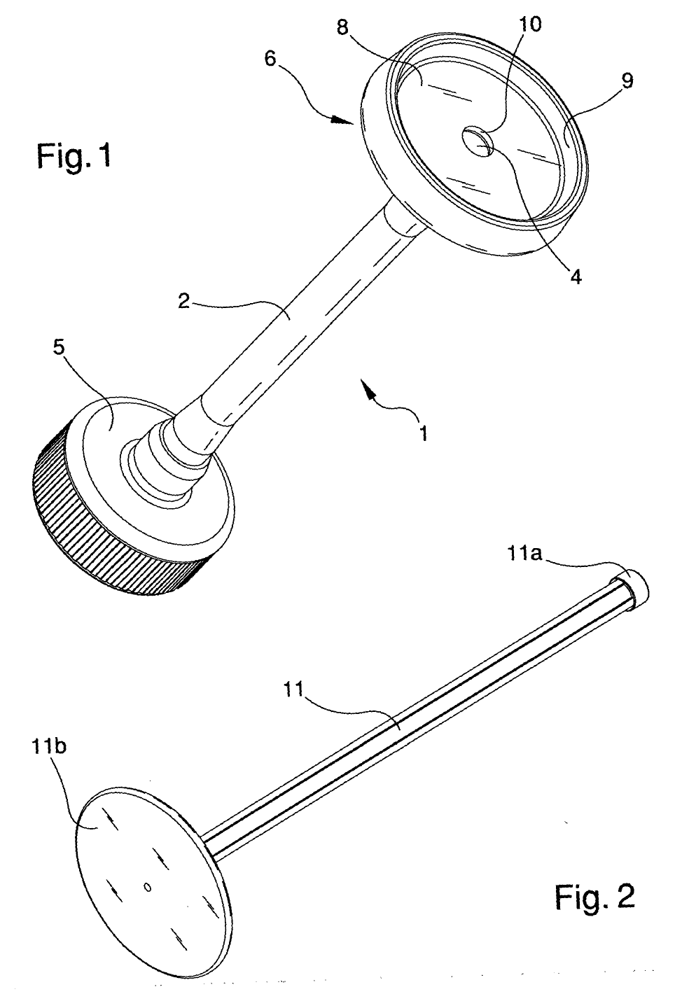

FIG. 1 is a perspective view of the transfer channel and of the applicator element as provided by the device according to the invention;

FIG. 2 is a perspective view of the thrust piston as provided by the device according to the invention;

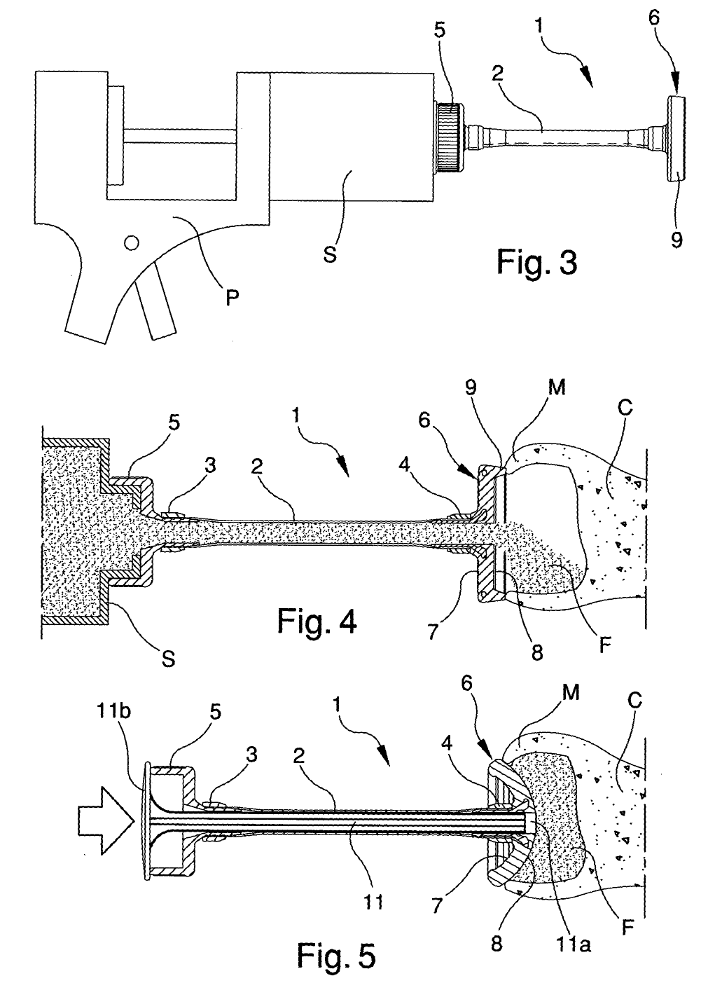

FIG. 3 is a side view of the transfer channel and of the applicator element as provided by the device according to the invention, applied to a fluid cement dispensing gun;

FIGS. 4 and 5 show, in a sequence of section views, a first mode of use of the device according to the invention;

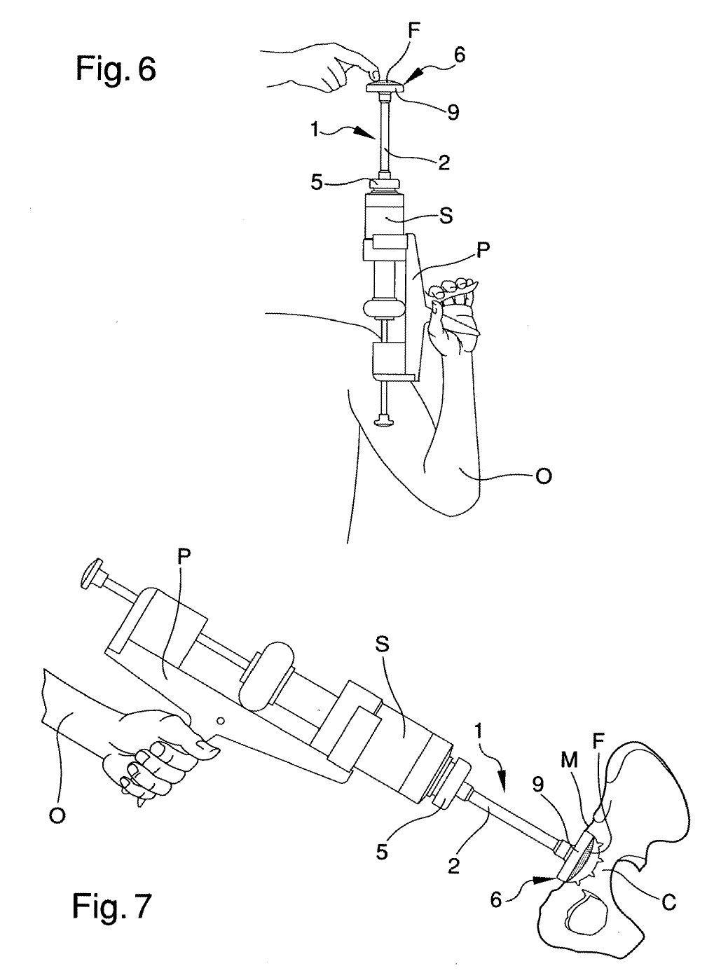

FIGS. 6 to 9 show, in a sequence of side views, schematic and partial, a second mode of use of the device according to the invention.

With special reference to such figures, a device for filling bone cavities with fluid cement, acetabular cavities in particular, has been generally designated by reference number 1.

The device 1 comprises a transfer channel 2 for fluid cement F, such as metacrylate resin type bone cements, which can be radiopaque or of other similar substances.

The transfer channel 2 is substantially rectilinear and tubular in shape and has a first opening 3 obtained at one end of the channel, and a second opening 4 obtained at the opposite end.

The first opening 3 is associable to a dispenser instrument for dispensing the fluid cement F, e.g. of the type of a syringe S, eventually fitted on a gun P, of the type already known.

In detail, at the end of the transfer channel 2 where the first opening 3 is obtained are provided connection means 5 for connecting to the syringe S. Such connection means are composed of a cylindrical fitting, coaxial to the transfer channel and associable to the syringe S by means of a removable coupling, e.g. of the threaded type, interlocking type or the like.

In practice, once the cylindrical fitting 5 is attached to the body of the syringe S the fluid cement F is pushed along the transfer channel 2 by means of the operation of the gun P and comes out of the second opening 4.

Usefully, an applicator element 6 is associated by the second opening 4 for injecting the fluid cement F inside a bone cavity C, for example of the acetabular bed type inside which hip prosthesis, or the like, are fitted.

In detail, the applicator element 6 has a useful dispensing surface larger than the transfer channel 2. By useful surface we mean the area through which the fluid cement F flows by the channel and the element.

The applicator element 6 comprises a first connecting side 7 for connecting to the transfer channel 2 and a second application side 8 for applying the fluid cement F.

The second side 8 has a perimeter containing border 9 for the fluid cement F, which can be placed in contact with the edge M of the bone cavity C, that is, against the outer border of the cavity.

In the particular embodiment of the invention, the applicator element 6 is made of a circular disk-shaped plate-like body that extends substantially at right angles with respect to the transfer channel 2.

The main opposing surfaces of the plate-like body 6 define the first and second sides 7 and 8.

The plate-like body 6 has a through hole 10 in which the fluid cement F flows which can be coupled to the end of the transfer channel 2 which ends with the second opening 4.

In detail, the through hole 10 is obtained on the plate-like body 6 in a substantially central position and the transfer channel 2 and applicator element 6 are coaxially associated together.

When using, the containing border 9 of the applicator element 6 is placed against the edge M of the bone cavity C so as to isolate the inside of the bone cavity C from the outside and so it can be filled with the fluid cement F without any leaks.

In detail, the containing border 9 consists of a perimeter turned-up edge of the plate-like body 6, extending substantially crossways from the second side 8 of the body itself.

Alternative embodiments are not to be ruled out, wherein the applicator element 6 is completely flat and the containing border 9 is composed of the outline of the plate-like body 6.

Usefully, the device 1 has thrusting means 11 that allow the fluid cement F to be pressed along the transfer channel 2 to ensure it is emptied at the end of the implant.

Such thrusting means are made up of a longitudinal piston with an end head 11a that can be substantially fitted to measure in the transfer channel 2, and a gripping handle 11b arranged opposite the end head 11a.

After a certain quantity of fluid cement F has been injected into the bone cavity C the syringe S has to be taken off the device 1 and the piston 11 inserted in the transfer channel 2 in order to finish filling the cavity.

The piston 11 is at least as long as the transfer channel 2; in this way, once the piston 11 is inserted in the channel 2 through the first opening 3, the end head 11a reaches the second opening 4 when the gripping handle 11b is against the cylindrical fitting 5.

Advantageously, the applicator element 6 is made in a flexible material while the transfer channel 2 is made in a substantially rigid material; in this way, after the fluid cement F has been dispensed, the device 1 can be used to compress the fluid, making it adhere to the inner walls of the bone cavity C.

By exerting a certain pressure on the transfer channel 2 and on the piston 11 inside it, actually, the plate-like body 6 is pressed against the fluid cement F deforming it elastically and turning the fluid cement F into a hollow configuration, suitable for receiving a semi-spherical prosthesis like, for instance, the cotyle of a hip prosthesis.

It should also be pointed out that the rigidity of the transfer channel 2 allows the fluid cement F to be pressurised also when it is being dispensed by the syringe S by the simple operator's manual pressure on the gun P lengthways to the channel itself.

It cannot, however, be ruled out that the transfer channel 2 can alternatively consist of a flexible pipe which, when dispensing the fluid cement F using the syringe S, is held manually in position by the bone cavity and, when compacting the fluid cement F in the cavity, becomes rigid when the piston 11 is placed inside it.

The device 1 according to the invention can be subject to different modes of use.

One first mode of use is illustrated in FIGS. 4 and 5 and sees the fluid cement F being dispensed through the transfer channel 2 only after the applicator element 6 has been positioned with the containing border 9 resting against the edge M of the bone cavity C.

In this mode of use, the space delimited by the containing border 9 and by the bone cavity C is initially free and the fluid cement F flows into it, pushed through the transfer channel 2 by the operation of the gun P (FIG. 4).

Once the bone cavity C is filled, the syringe S can be detached from the transfer channel 2 and the piston 11 can be inserted in the transfer channel 2 so the device 1 is pressed towards the bone cavity C.

The bending of the plate-like body 6 inside the bone cavity C allows the fluid cement to be pressurised and get it to penetrate in depth in the bone tissue (FIG. 5).

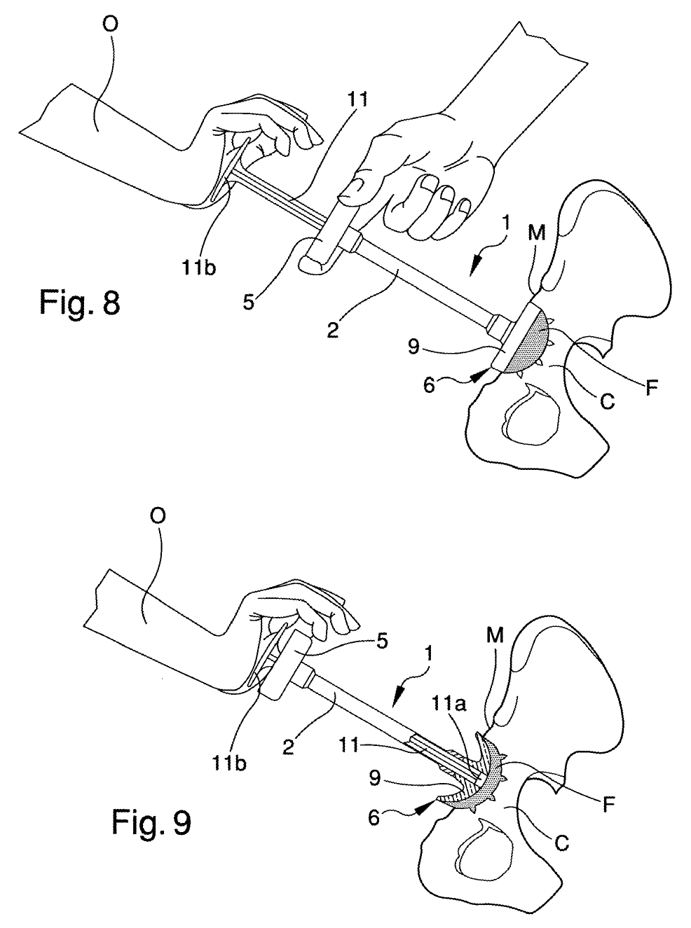

In a second mode of use of the device 1, illustrated in figures from 6 to 9, the procedure for filling bone cavities with fluid cement, acetabular cavities in particular, comprises the phases consisting in:
supplying the device 1 and the dispenser instrument S, separated one from the other, and mounting the device 1 on the dispenser instrument S;
positioning vertically the gun P that carries the dispenser instrument S so as to arrange the applicator element 1 with the second side 8 facing upwards;
dispensing the fluid cement F through the transfer channel 2 until the fluid cement F comes on the surface of the applicator element 6 and filling, at least partially, the volume surrounded by the containing border 9. Preferably this phase is carried out by the medical operator O using the gun P and finished when the fluid cement F protrudes slightly from the containing border 9;
leaving the fluid cement F on the applicator element 6 to harden until the fluid has reached a pre-established viscosity. This phase entails the operator O regularly measuring the viscosity of the fluid cement F, e.g. feeling with his fingers the fluid cement F that comes on the surface of the applicator element 6 (FIG. 6). This is done wearing sterilised gloves that protect the hands of the operator O and preventing any contamination of the fluid cement F;
resting the containing border 9 on the edge M of the bone cavity C (FIG. 7);
pushing the fluid cement F inside the bone cavity C. Initially this phase consists in operating the dispenser instrument S by means of the gun P until the fluid cement F can be seen on the borders of the applicator element 6. Subsequently it consists in separating the dispenser instrument S from the transfer channel 2, leaving the device 1 in position, and inserting the piston 11 in the transfer channel 2, so as to pressurise the fluid cement F inside the bone cavity C (FIG. 8). Lastly, this phase consists in pressing the device 1 repeatedly towards the bone cavity C so as to bend the plate-like body 6 inside the bone cavity C and to make the fluid cement F penetrate the bone tissue in depth (FIG. 9).

It has in fact been found that the described invention achieves the intended objects.

It should be noted that the particular type of applicator element provided in this invention allows the fluid cement to be channeled directly into the bone cavity through the syringe without the risk of unwanted or sudden leaks of fluid or of dangerous contamination.

Also note that with this invention not only is it possible to apply the fluid cement easily and practically but it can also be compacted quickly either during injection or at the end of dispensing.

Lastly, we would like to point out that having a containing border like the one illustrated in this invention, the operator is able to keep a small quantity of fluid cement to control viscosity and fill the bone cavity with it only when the moment is right.

The invention thus conceived is susceptible of numerous modifications and variations, all of which falling within the scope of the inventive concept. Furthermore all the details can be replaced with others that are technically equivalent.

In practice, the materials used, as well as the shapes and dimensions, may be any according to requirements without because of this moving outside the protection scope of the following claims.

1. Device for filling bone cavities including acetabular cavities with fluid cement, comprising at least one transfer channel for transferring a fluid cement with at least one first opening which is associated with a dispenser instrument for dispensing said fluid cement and one second opening through which said fluid cement exits, and at least one applicator element of said fluid cement to a bone cavity which can be associated with said transfer channel by said second opening and has a dispensing surface that is larger than said transfer channel, wherein said applicator element comprises a substantially plate-like body whose main opposite surfaces define a first connecting side to said transfer channel and a second application side of said fluid cement, said second side comprising a containing border for containing said fluid cement which can be positioned in contact against the edge of said bone cavity and which comprises a perimeter turned-up edge of said plate-like body which extends substantially crosswise from the body itself.

2. Device according to claim 1, wherein said plate-like body has a through hole in which said fluid cement flows which is coupled to said second opening.

3. Device according to claim 2, wherein said hole is obtained in said plate-like body in a substantially central position.

4. Device according to claim 1, wherein said plate-like body is substantially circular.

5. Device according to claim 1, wherein said transfer channel and said plate-like body are associated substantially at right angles.

6. Device according to claim 1, wherein said transfer channel and said applicator element are substantially coaxially associated together.

7. Device according to claim 1, wherein said applicator element is made in a substantially flexible material.

8. Device according to claim 1, wherein said transfer channel is made in a substantially rigid material.

9. Device according to claim 1, wherein said transfer channel comprises connection means to said dispenser instrument arranged by said first opening.

10. Device according to claim 9, wherein said connection means comprises at least one fitting substantially coaxial to said transfer channel.

11. Device according to claim 1, wherein the device comprises thrusting means of the fluid cement in said transfer channel.

12. Device according to claim 11, wherein said thrusting means comprises at least one piston which can be fitted to measure in said transfer channel.

13. Device according to claim 12, wherein said piston is at least as long as said transfer channel.

14. Procedure for filling bone cavities with fluid cement including acetabular cavities, comprising:
supplying a device as recited in claim 1, mounted on said dispenser instrument;
positioning said applicator element with said second side facing upwards;
dispensing said fluid cement through said transfer channel until filling, at least partially, a volume defined by said containing border;
leaving said fluid cement on said applicator element to harden until the fluid has reached a pre-established viscosity;
resting said containing border on the edge of said bone cavity; and
pushing said fluid cement inside said bone cavity.

15. Procedure according to claim 14, wherein said supplying comprises supplying said device and said dispenser instrument separated one from the other, and mounting said device on said dispenser instrument.

16. Procedure according to claim 14, wherein said dispensing finishes when said fluid cement protrudes partially from said containing border.

17. Procedure according to claim 14, wherein said leaving the fluid cement to harden comprises measuring the viscosity of said fluid cement.

18. Procedure according to claim 17, wherein said measuring comprises feeling said fluid cement that comes on the surface of said applicator element.

19. Procedure according to claim 18, wherein said feeling is done with fingers.

20. Procedure according to claim 14, wherein said pushing comprises operating said dispenser instrument.

21. Procedure according to claim 14, wherein said pushing comprises inserting said thrusting means in said transfer channel.

22. Procedure according to claim 14, wherein said pushing comprises pressing said device towards said bone cavity so as to bend said applicator element inside the bone cavity.
